(19) United States
(12) Patent Application Publication
(10) Pub. No.: US 2003/0130386 A1
(43) Pub. Date: Jul. 10, 2003

(54) Hydrazine-based diurea derivatives

(76) Inventors: Richard Hendrikus Gerrit Brinkhuis, Zwolle (NL); Petrus Johannes Maria David Elfrink, Boxmeer (NL)

Correspondence Address: MICHELLE J. BURKE, AKZO NOBEL INC., 7 LIVINGSTON AVENUE, DOBBS FERRY, NY, 10522-3408, US

(21) Appl. No.: 10/279,275

(22) Filed: Oct. 24, 2002

Related U.S. Application Data
(60) Provisional application No. 60/333,932, filed on Nov. 28, 2001.

(30) Foreign Application Priority Data
Oct. 26, 2001 (EP) 01204060.6

(51) Int. Cl. C07C 281/06; C08K 5/22; C08K 5/24

(52) U.S. Cl. 524/189; 564/35

(57) ABSTRACT

Acicular hydrazine-based diurea derivative crystals of the formula R′HN—CO—NH—NH—CO—NHR wherein R and R′ independently are unsubstituted or substituted hydrocarbyl groups with an average length of the crystals ranging from about 0.1 to about 300 micrometers, and the use of such crystals as a rheology modifier in coatings and adhesives. Preferably, the hydrocarbyl group is a hexyl, octadecyl, benzyl, or cyclohexyl group.

This application claims priority of European Patent Application No. 01204060.6, filed Oct. 26, 2001, and U.S. Provisional Patent Application No. 60/333,932, filed Nov. 28, 2001.

### FIELD OF THE INVENTION

The invention pertains to a hydrazine-based diurea derivative particulate, essentially formaldehyde-free compositions comprising said hydrazine-based diurea derivative, and the use of the hydrazine-based diurea derivative as a rheology modifier and anti-settling agent. The invention further pertains to coating and adhesive compositions comprising said hydrazine-based diurea derivative.

### BACKGROUND OF THE INVENTION

Thixotropic agents or sag control agents are essential elements of coating and adhesive compositions. They not only provide a stable mixture of the several components in a coating composition, but also enable the application of the coating or adhesive, even in thick layers on vertically placed objects, without the occurrence of sags in the cured coating. Colloidal associates of urea compounds are known to be useful in that they provide a thixotropic rheology, improving, e.g., the sag resistance of coatings (hence the term sag control agent, SCA) or the orientation of metallic pigments in a base coat for automotive coatings and improving the appearance of coatings applied on vertical surfaces. Amine-based diurea derivatives are well-known sag control agents and are described in numerous patents. Known amine-based diurea derivatives are amino-functional compounds based on primary amines such as benzylamine, methylamine, hexylamine, 1,6-hexamethylenediamine, neopentyldiamine, and the like.

EP-A-0 198 519 discloses diurea compounds, the adducts of hexamethylene-1,6-diisocyanate (HDI) to benzylamine (BA), which are used in industry for the above purposes and are characterized by an anisotropic particle shape. The anisotropy is considered to allow for the formation of a percolating network of SCA particles upon flocculation (leading to a strong viscosity increase) at lower volume fractions than would be the case with isotropic particles. SEM pictures of HDI-BA diurea adducts reveal that the ratio of the longest to the shortest dimension (aspect ratio) is typically around five. The average length of these HDI-BA particles is 2-3 microns.

There have been substantial efforts to develop other compounds that can function as an SCA in coating (paint) and adhesive compositions.

### SUMMARY OF THE INVENTION

We have found that specific alternative diurea compounds, namely those prepared from hydrazine (H₂N—NH₂) and mono-isocyanates, can be produced such that needle-like self-associated particles are formed that can or may show extreme anisotropy (aspect ratios over 300 in some cases). These needle-like particles were found to be more effective in inducing thixotropic rheology in coating formulations, at very low concentrations, than the existing di, tri and polyurea SCAs. The diurea compounds according to the invention can be used in resins for coatings with an improved appearance on vertical surfaces. They exhibit a broader application window than the known SCAs. In this context the term “application window” means the limits of layer thicknesses between which a coating or adhesive can be applied onto a vertical surface without sagging and with sufficient leveling for automotive and adhesive applications.

It is noted that the chemical compounds as such, obtained by, for example, reacting a mono-isocyanate and hydrazine, are well-known in the art. These products are known to be highly efficient scavengers of formaldehyde, for which purpose they have typically been used. The hydrazine-based diurea derivatives are consumed by said scavenging action. The physical form of the hydrazine-based diurea derivative is unimportant in this application.

### DETAILED DESCRIPTION OF THE INVENTION

The present invention relates to specific compositions comprising hydrazine-based diurea derivatives of the formula R′HN—CO—NH—NH—CO—NHR, wherein R and R′ independently are unsubstituted or substituted hydrocarbyl groups. These compositions are characterized in that they comprise the hydrazine-based diurea derivative in the form of acicular crystals, so that they function as an SCA, and in that the compositions are essentially free of formaldehyde or compounds that generate formaldehyde.

Preferably, the hydrocarbyl group is substituted or unsubstituted and selected from an alkyl, cycloalkyl, aralkyl, and aryl group. More preferably it is a substituted or unsubstituted monovalent aliphatic hydrocarbon group having 1 to 18 carbon atoms, a monovalent cycloaliphatic hydrocarbon group having 5 to 15 carbon atoms, a monovalent araliphatic hydrocarbon group having 7 to 15 carbon atoms, or a monovalent aromatic hydrocarbon group having 6 to 15 carbon atoms. Even more preferably, it is selected from methyl, butyl, hexyl, octadecyl, benzyl, methoxybenzyl, phenyl, methoxyphenyl, naphthalene, biphenyl, methoxypropyl, ethoxypropyl, methacryloyl-C₁₋₈ alkyl, preferably methacryloyl-C₁₋₄ alkyl, most preferably methacryloylethyl, with the formula

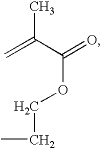

acryloyl-C₁₋₈ alkyl, preferably acryloyl-C₁₋₄ alkyl, most preferably acryloylethyl and mixtures thereof. Most preferred are the hydrocarbyl groups selected from hexyl, octadecyl, methoxypropyl, ethoxypropyl, benzyl, cyclohexyl,

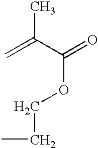

and combinations thereof. It is also preferred that R and R′ are the same. Of these hydrazine-based SCAs, the compounds prepared by reacting hydrazine and cyclohexyl isocyanate, octadecyl isocyanate, or a mixture thereof, the hydrocarbyl groups therefore being cyclohexyl, octadecyl, and mixtures thereof, are especially preferred.

The acicular form of the crystals can be characterized by the aspect ratio, i.e. the length (longest dimension) of the crystals divided by their width, as can be determined by (visual) inspection of SEM pictures. This aspect ratio should be more than 3, preferably more than 10, most preferably more than 20. Another important characteristic is the average length (le) of the hydrazine-based diurea derivative particles, as can be determined by (visual) inspection of SEM pictures. Depending on the conditions chosen when making the hydrazine-based diurea derivative, the le can vary from 0.1 to 300 micrometres (microns). Suitably the conditions are chosen such that the le is more than 0.5, preferably more than 1, more preferably more than 2, and most preferably more than 3 microns. The lower numbers are less preferred, since compositions comprising the hydrazine-based diurea derivative with such an le may still suffer from sagging or may require high loads of the SCA. In order to control the viscosity of compositions in which the SCA is used, the le preferably is less than 100, more preferably less than 50, and most preferably less than 30 microns. If the viscosity of a composition comprising a hydrazine-based diurea derivative according to the invention is too high, the le can be reduced or the amount of the hydrazine-based diurea derivative can be decreased, and vice versa.

The term “hydrazine” means hydrazine, hydrazine hydrate or salts thereof. Preferably, aqueous solutions of hydrazine are used.

The term “mono-isocyanate-functional compounds” is used to denominate isocyanates that contain only one amine-reactive isocyanate group per molecule. The term “essentially free of formaldehyde or compounds that generate formaldehyde” is used here to denote compositions that do not contain or generate enough formaldehyde during regular processing to completely consume the SCA. Preferably, the compositions are completely free of formaldehyde during their processing, because then no SCA will be consumed before the final coating or adhesive composition is applied or formed, ensuring the full sag control effect of the SCA that was introduced. However, for two-component coating or adhesive formulations it can be advantageous to use a small amount of a formaldehyde-based resin or a compound that generates formaldehyde in order to lower the curing temperature and to obtain a clear, haze-free coating in the case of clear coats. In that case, the amount of formaldehyde formed during processing and curing of the coating or adhesive compositions preferably is less than 100%, more preferably less than 50%, even more preferably less than 25% of the amount that can be scavenged by the hydrazine-based diurea derivative present in the formulation.

The formaldehyde-based resin or compound that generates formaldehyde during the curing conditions is preferably added just before application or it can be added to the component of the two-component coating which does not contain the hydrazine-based diurea derivative.

The term “essentially acicular” is meant to denominate crystals with a length in one dimension that is at least 2 times, preferably 4 times, more preferably at least 6 times, and most preferably at least 8 times, the length in any other dimension. Typically, the crystals are needle-shaped.

The term “adhesive” is meant to denominate cements, glues, sealants, as well as putties.

Conventional sag control agents are known to have been produced in the presence of a binder resin. In GB 1,454,414, for example, a urea adduct is prepared in situ in the presence of the binder. Also the hydrazine-based diurea derivatives according to the invention are preferably produced in the presence of a binder so as to directly form needles of the desired le in the binder resin. However, they can also be produced by reacting hydrazine and isocyanate in a non-solvent with a limited solubility for the hydrazine-based diurea derivative, resulting in a precipitate that can be used as such, be milled in order to reduced the le, or be recrystallized, e.g., to increase the purity or to change the le. In the process of making the precipitate of the hydrazine-based diurea derivative the dosing conditions can be varied and the stirrer speed changed in order to change the le of the resulting needles. Such optimizations are known to the skilled person.

Alternatively, the hydrazine-based diurea derivative can be prepared as a true solution at temperatures between 0 and 150° C. The needles of the hydrazine-based diurea derivative can subsequently be precipitated from the solution using conventional techniques, such as the addition of or addition to a non-solvent, and then mixed with the binder. However, if the solvent is selected such that it has affinity for the binder, the solution can be combined with said binder and form the precipitate directly in said binder. Suitable solvents for this purpose are, for example, N-methyl pyrrolidone (NMP) or butylglycol.

These known methods are equally applicable with the present invention. Usually, hydrazine or a hydrate or a salt thereof is reacted with at least one mono-isocyanate of the formula R—NCO or R′—NCO, wherein R and R′ have the previously given meanings, in one of the above-disclosed manners. Also mixtures of mono-isocyanates of formulae R—NCO or R′—NCO can be used. The reaction between the hydrazine and the mono-isocyanate-functional compound can generally be carried out in any arbitrary sequence of combining the starting compounds, optionally at elevated or lowered temperature. It is preferred that the reaction should be carried out in an atmosphere of an inert gas at temperatures in the range of −20 to +80° C. The reaction can be performed with mixtures of mono-isocyanates R—NCO and R′−NCO, wherein R and R′ have a different meaning, or with just one isocyanate of the formula R—NCO.

If the overall amount of hydrazine and the isocyanate-functional compound is stoichiometric, then each amine group reacts with an isocyanate group of the isocyanate-functional compound, such that essentially no free amine or isocyanate groups are present after completion of the theoretical reaction. However, in order to prevent the presence of small amounts of unreacted amino groups of the hydrazine, a slight excess of isocyanate-functional compound is preferred. Preferably, this excess ranges from approximately 1 to 10%, based on equivalents.

In accordance with a preferred embodiment, the thixotropic resin with the hydrazine-based diurea derivative is obtainable by adding a mono-isocyanate-functional compound to a mixture of hydrazine and resin, resulting in anisotropic acicular crystals embedded in the resin. However, the hydrazine and the mono-isocyanate-functional compound can be added to the resin in any sequence. If so desired, this may be done in several steps. If the mono-isocyanate-functional compound is added first, care should be taken that the thus obtained mixture is not stored for too long or at a too high temperature, since a reaction between the resin and the mono-isocyanate-functional compound might occur. It is also possible to add the amine compound to a portion of the resin and to add the isocyanate-functional compound to another portion of the resin, followed by combining the two resin mixtures, optionally with additional resin being added.

Alternatively, the hydrazine-based diurea derivative can be made in a coating formulation instead of in a mere resin using any sequence described above.

During the reaction of the amine and the isocyanate-functional compounds the reaction mixture is preferably thoroughly mixed. Suitably this is done by using low- or high-shear mixers (such as propellers, impellors, turbines, rotor-stator mixers, dispersers, dissolvers, and the like).

Additionally, if a precipitate is formed, it can be preferable that one or more additives which function as a crystal growth modifier and/or flocculation modifier for the SCA particles are present.

When crystal growth modifiers are used, the le of the SCA needles can be influenced. One or more crystal growth modifiers can be added before or simultaneously with the formation of the hydrazine-based diurea derivative. Alternatively, one or more of the crystal growth modifiers are formed during the crystallization step of the hydrazine-based diurea derivative. Preferably, the crystal growth modifiers comprise at least one polar, preferably “ureaphilic,” i.e. multiple hydrogen bridging group, with the proviso that the crystal growth modifiers are different from the main crystallizing diurea compound. Such molecules will interact with the hydrazine-based diurea derivative that is formed, retarding further crystallization of the nucleus Preferred ureaphilic groups are urea, amide, and urethane groups. Whether or not a compound with ureaphilic groups is an effective crystal growth modifier is easily determined by carrying out the precipitation step of the hydrazine-based diurea derivative both with and without 2% by weight of said component (based on the hydrazine-based diurea derivative) and to judge the differences in aspect ratio and/or length of the needles that are formed.

Flocculation modifiers can be added before, simulataneously with, or after the formation of the hydrazine based diurea derivative. When flocculation modifiers are used, the stabilization of the SCA crystals towards flocculation can be enhanced, and flocculation retarded. Such additives are characterized by a molecular weight of at least 500 Dalton, more preferably, the additives are selected from molecules with a molecular weight of at least 600 Dalton, even more preferably at least 800 Dalton, more preferably still at least 1,000 Dalton, and most preferably more than 2,000 Dalton, and comprise a polar (preferably ureaphilic) moiety as described above, as well as a moiety, such as a main chain, that has good compatibility with the resin/solvent in which the precipitate is formed. Such compounds may have both a crystal growth modifier as well as a flocculation modifier function. Flocculation retardation can be observed as a (relative) slowing down of the structure build-up after high shear treatment in a rheological creep test. Suitable modifiers are molecules with one or more urea groups such as are for example obtainable by reacting an isocyanate with a bulky or long-chain amine, such as Jeffamine M1000.

The preparation of the hydrazine-based diurea derivative can be performed batchwise as well as in a continuous process.

The amount of SCA to be used in a binder concentrate, a coating composition or an adhesive composition can vary over a wide range, depending on the circumstances. In a preferred embodiment, the SCA according to the invention is present in a concentrated resin composition. Such concentrates have particular advantages when making the coating or adhesive compositions. More specifically, they ensure great flexibility and dosing accuracy of the SCA. At present it is expected that the concentrates will contain approximately from 0.1 to 50% by weight of the SCA, based on the weight of the total concentrate (%w/w). Preferably, the amount of SCA in the concentrate is greater than 0.1, more preferably greater than 0.5, even more preferably greater than 1.0, and most preferably greater than 1.5%w/w. It is preferred that the concentrate comprises less than 50, more preferably less than 25, and most preferably less than 10%w/w of the SCA. Especially since these concentrates are typically stored for a prolonged period of time before they are used in making the coating or adhesive formulation, it is preferred that they are formaldehyde-free.

The amount of SCA present in the coating or adhesive formulation depends, inter alia, on the rheology of the formulation in which it is used and the sag control required. It is noted that an SCA according to the invention can be combined with other rheology control agents such as conventional SCA based on HDI-BA such as described, for example, in EP-A-0 192 304. Especially when the curing temperature of the final coating or adhesive is over 100° C., such a combination with conventional SCAs can be advantageous. Typically, the total SCA content originating from both types of SCA is between approximately 0.1 and 5%. Alternatively, the hydrazine-based diurea derivative can be combined with non-aqueous dispersions or microgels in coating formulations in order to optimize the rheological behaviour and performance of coatings.

The thixotropic resins according to the present invention are pre-eminently suited to be used in the formulation of coating and adhesive compositions. In coating compositions these resins can be used as a functional binder in combination with cross-linkers, such as cross-linking resins, e.g., for curing in 1K- and 2K-coating compositions at room temperature, forced drying conditions (30-100° C.), and baking conditions (higher than 100° C.). Forced drying and baking conditions are preferred. High-temperature baking is preferably performed at 100-160° C. Furthermore, the present rheology modifiers have excellent sag resistance at lower concentrations than the known rheology modifiers, such as SCAs based on di-, tri-, or polyisocyanates with mono-amines.

It should be noted that the cross-linker used in the final coating or adhesive formulation can be of any conventional type suitable for the resin used, as long as the formulation is essentially formaldehyde-free. Known resin/cross-linker and/or cross-linker/resin combinations in which the SCA according to the invention can be used include: polyol or thiol/(blocked) polyisocyanate; epoxy/acid combinations; epoxy/anhydride combinations; epoxy/acid-anhydride; acetoacetate/amine, aldehyde blocked amine, or ketone blocked amine; acryloyl/amine, aldehyde blocked amine, or ketone blocked amine; epoxy/thiol.

Polyols include acrylic polyols, polyester polyols, polyether polyols, silicon-containing polyols, urethane modified polyols and (internally) blocked polyols, and mixtures thereof.

However, the SCA according to the invention can also be used in compositions where curing is obtained by means of actinic radiation, such as near-infra red, visible light, UV and X-ray radiation or electron beam radiation. Such curing processes can involve mere curing through unsaturated covalent bonds, such as ethylenically unsaturated carbon-carbon bonds, but any other conventional reactive bond may be involved in the curing process as well. Also, it can be beneficial to use a conventional radiation-susceptible activator/initiator in such curing processes. Examples of suitable radiation-curable compositions are presented in WO 02/18468. The conventional SCA as used therein can be replaced by the SCA according to the invention.

It was found that any resin common in coatings and adhesives can be used in the present invention. Examples are polyesters, ester diols, polyurethanes, alkyd resins, acrylate and methacrylate resins, epoxy resins, polyamine, carbamate, and polyester acrylate resins, and the like. These resins may optionally be functionalized with hydroxy, keto, carboxylate, sulfonate, carbamate, epoxy groups, tertiary amines, and the like. Also, the final coating or adhesive composition can be solvent or water borne.

The thixotropic coating composition can further contain the usual adjuvants and additives, for instance pigment dispersants, dyes, pigments, solvents, and accelerators for the curing reaction.

The thixotropic coating composition can be applied to a substrate in any desired manner, for instance by rolling, spraying, brushing, sprinkling, casting, dipping or electrostatic spraying. The thixotropic coating composition can further be cured or baked in the usual way, for instance at ambient temperatures or in an oven at higher temperatures, for instance in the range of approximately 60 to 140° C., over a period of 2 to 120 minutes.

The invention is further illustrated by the following examples:

### EXAMPLE 1

2.96 g of a hydrazine solution (35 wt. % in water) were added to 300 g of a polyester polyol resin (Setal® 166 SS-80, 80% solids in butyl acetate) at room temperature. Under stirring, a solution of 8.08 g of cyclohexyl isocyanate (CHI) was added over a period of 2 min. Stirring was continued for 5-10 min. A turbid colloidal suspension with needles of the reaction product (HY/CHI) of hydrazine (HY) and cyclohexyl isocyanate (CHI) having an le of about 60-70 microns with strong thixotropy was obtained.

### EXAMPLE 2

Into a 2-litre reaction vessel equipped with a high-speed dissolver and two dropping funnels were charged 600 g of Setal® 166 SS-80 (a polyester polyol resin, ex Akzo Nobel) and 138.5 g of butyl acetate. The contents of the reaction vessel were mixed at 1,700 rpm. Then 5.84 g of a 35% aqueous hydrazine solution were added and mixed at 1,700 rpm for 5 minutes. Next a mixture of 16.44 g of cyclohexylisocyanate and 16.44 g of butyl acetate was added in 6 min at 3,000-3,550 rpm. After the addition, the reaction mixture was stirred for another 10 min at 4,000-4,350 rpm. The obtained white solution had a theoretical percentage of 2.32 of the hydrazine-based diurea compound. The viscosity at 1 and 100 s⁻¹ amounted to 14.1 and 0.64 Pas, respectively.

### EXAMPLE 3

6.23 g of an adduct of Jeffamine® M1000 to cyclohexylisocyanate (CHI) (1:1 molar), which acts as a crystal growth inhibitor, were added to 150 g Setal® 166 solution (80% solids); 1.47 g of hydrazine (35 wt. % aqueous solution) were added, and under stirring 4.02 g of CHI were added over 2 min. Stirring was continued for 5-10 min, and a turbid colloidal suspension with strong thixotropic rheology was obtained.

SEM analysis of the colloidal particles formed in the resin revealed the extreme aspect ratio (long thin needles).

### EXAMPLE 4

138.46 g of an adduct of Jeffamine® M1000 to CHI (1:1 molar) were added to 600 g of a polyester polyol resin (Setal® 166, 80% solids) at room temperature followed by the addition of 5.84 g of hydrazine solution (35 wt. % in water). Under stirring, a solution of 16.44 g of CHI in 16.44 g of butyl acetate was added over a period of 6 min. Stirring was continued for 10 min. A turbid colloidal suspension with strong thixotropy was obtained. The fineness of the urea suspension was shown to be <8 μm (using a Hegman bar).

### EXAMPLE 5

0.71 g of a hydrazine solution (35 wt. % in water) was added to 150 g of a polyester polyol resin (Setal® 166, 80% solids) at room temperature. Under stirring, a solution of 4.27 g of octadecyl isocanate (ODI) was added over a period of <2 min. Stirring was continued for 5-10 min. A thixotropic turbid colloidal suspension containing needles was obtained.

### EXAMPLE 6

Into a 2-litre reaction vessel equipped with a high-speed dissolver and two dropping funnels were charged 400 g of Setal® 166 SS-80 (a polyester polyol resin, ex Akzo Nobel) and 92.3 g of butyl acetate. The contents of the reaction vessel were mixed at 1,700 rpm. Then 3.89 g of a 35% aqueous hydrazine solution were added and mixed at 1,700 rpm for 5 minutes. Next a mixture of 10.96 g of cyclohexyl isocyanate and 10.96 g of butyl acetate was added in 6 min at 4,500-4,800 rpm. After the addition, the reaction mixture was stirred for another 20 min at 14,000 rpm.

The obtained white solution had a theoretical percentage of 2.32 of the hydrazine-based diurea compound. The viscosity at 1 and 100 s⁻¹ amounted to 15.5 and 0.66 Pa.s, respectively. The needles had an le of 10 to 50 microns.

### EXAMPLE 7

A lacquer was formulated by adding 1.38 g of the product of Example 3 to 3.15 g of Setal® 166 (80% solids). Stirring was continued until a homogeneous mixture was obtained. Under stirring, 1.12 g of butyl acetate were added slowly over a period of 510 ? min. To this homogeneous mixture 2.29 g of Tolonate® HDT-LV were added (total 0.67 wt. % diurea on solids, solids content 75%). Stirring was continued for 5 min. The rheology of the lacquer was measured and showed a fast recovery over more than two decades (75 Pa.s after 200 sec at 0.5 Pa shear stress after disturbing at 1,000 s⁻¹; high-shear viscosity 0.5 Pa.s.

The lacquer was applied to a glass substrate with a doctor blade at a film thickness of 120 μm, and cured for 30 min at 90° C. A clear glossy film was obtained: no turbidity of colloidal particles was visible.

### EXAMPLE 8

In a first series two paints A (according to the invention) and B (comparative) were prepared according to Table I. The paints were sprayed onto a vertical tin plate with 13 holes of 1 cm diameter and vertically baked at the indicated schedules. The sagging limit was determined as the layer thickness between the hole at which sagging occurs and the preceding hole. Another way to measure sagging is to measure the layer thickness at the point where the length of the tear is 1 cm. The resin of Example 2 is more effective than Setal® 80166 SS-64. The higher the sagging limit, the more effective the SCA is!

TABLE I

| | A | B |
|---|---|---|
| Setal ® 166 SS-80 (solids) | 43.0 | 43.0 |
| Resin of Example 2 (solids) | 18.2 | — |
| Setal ® 80166 SS-64 (solids) | — | 18.2 |
| Silicon oil L050 (1% in butyl acetate) | 5.0 | 5.0 |
| Tinuvin ® 292 | 0.5 | 0.5 |
| Tinuvin ® 1130 | 1.0 | 1.0 |
| Tinstab ® BL 277 (1% in butyl acetate) | 0.7 | 0.7 |
| Tolonate ® HDT 90 (solids) | 38.8 | 38.8 |
| Butyl acetate to 23 sec DIN cup 4/23° C. | | |
| % SCA | 0.68 | 0.68 |
| Forced drying for 45 min at 80° C. | | |
| Sagging limit (micron) | 61 | 48 |
| Tear 1 cm (micron) | 88 | 68 |
| Baking for 24 min at 140° C. | | |
| Sagging limit (micron) | 53 | <41 |
| Tear 1 cm (micron) | 71 | 54 |

### EXAMPLE 9

In a second series (see Table II) again two paints C (according to the invention) and D (comparative) were prepared and applied. The sagging limit was measured as described in Example 8. Thus the effectiveness as anti-sagging agent at low SCA concentration becomes clear.

TABLE II

| | C | D |
|---|---|---|
| Setal ® 166 SS-80 (solids) | 43.0 | 61.2 |
| Resin of Example 2 (solids) | 18.2 | — |
| Setal ® 80166 SS-64 (solids) | — | — |
| Silicon oil L050 (1% in butyl acetate) | 5.0 | 5.0 |
| Tinuvin ® 292 | 0.5 | 0.5 |
| Tinuvin ® 1130 | 1.0 | 1.0 |
| Tinstab ® BL 277 (1 % in butyl acetate) | 0.7 | 0.7 |
| Tolonate ® HDT 90 (solids) | 38.8 | 38.8 |
| Butyl acetate to 23 sec DIN cup 4/23° C. | | |
| % SCA | 0.68 | 0.0 |
| Forced drying for 45 min at 80° C. | | |
| Sagging limit (micron) | 56 | 28 |
| Tear 1 cm (micron) | 69 | 46 |
| Baking for 24 mm at 140° C. | | |
| Sagging limit (micron) | 43 | 26 |
| Tear 1 cm | 58 | 43 |

### COMPARATIVE EXAMPLE 10

162.1 g of Setal® 80166 (64% solids, 3.8% SCA (“clear” triurea type on solids) were added to 306.3 g of Setal® 166 (80% solids). Stirring was continued until a homogeneous mixture was obtained. Under stirring 72.15 g of butyl acetate were added slowly over a period of 5-10 min. Finally, 221.04 g of Tolonate® HDT-LV were added (solids content 75%, SCA on solids 0.68%). Stirring was continued for 5 min. The rheology was measured and compared with that of the lacquer from Example 6. At this concentration a very limited structural viscosity was built up after high shear disturbance (0.5 Pa.s after 200 sec at 0.5 Pa shear stress after disturbing at 1,000 s⁻¹; high-shear viscosity 0.5 Pa.s).

### COMPARATIVE EXAMPLE 11

Into a 2-litre reaction vessel equipped with a high-speed dissolver and two dropping funnels were charged 600 g of Setal® 166 SS-80 and 131.6 g of butyl acetate. The contents of the reaction vessel were mixed at 1,700 rpm. Then 10.08 g of benzylamine were added and mixed at 1,700 rpm for 5 minutes. Next a mixture of 8.15 g of benzylamine and 16.31 g of butyl acetate was added in 6 minutes at 3,000-4,500 rpm. After the addition, the reaction mixture was stirred for another 10 minutes at 3,800-4,000 rpm.

The obtained white solution had a theoretical percentage of 2.32 of HDI/BA SCA. The viscosity at 1 and 100 s⁻¹ amounted to 4.55 and 0.51 Pas, respectively.

### EXAMPLE 12

The following clear coat formulations were prepared. The clear coats were formulated in such a way that on solids all formulations contained the same amounts of acrylic resin and polyester resin. Examples A-C are comparative.

TABLE III

| Formulation | A | B | C | D | E | F |
|---|---|---|---|---|---|---|
| Setalux ® 1767 VV-65 | 34.1 | 34.1 | 34.1 | 34.1 | 34.1 | 34.1 |
| (solids) | | | | | | |
| Setal ® 166 SS-80 (solids) | 10.7 | 5.3 | | 10.7 | 5.3 | |
| Resin of example 11 - | 18.8 | 24.2 | 29.5 | | | |
| Setal ® 166 SS-80 modified | | | | | | |
| with HDI/BA (solids) | | | | | | |
| Setal ® 166 SS-80 modified | | | | 18.8 | 24.2 | 29.5 |
| with 3.75% (on solids) of | | | | | | |
| HY/CHI (solids) ¹ | | | | | | |
| Tinstab ® BL 277 (1% in | 0.9 | 0.9 | 0.9 | 0.9 | 0.9 | 0.9 |
| BuAc) | | | | | | |
| Baysilon ® OL-17 (1% in | 3.0 | 3.0 | 3.0 | 3.0 | 3.0 | 3.0 |
| BuAc) | | | | | | |
| BYK ® 306 | 0.5 | 0.5 | 0.5 | 0.5 | 0.5 | 0.5 |
| Tinuvin ® 292 | 0.5 | 0.5 | 0.5 | 0.5 | 0.5 | 0.5 |
| Tinuvin ® 1130 | 1.0 | 1.0 | 1.0 | 1.0 | 1.0 | 1.0 |
| Tolonate ® HDT 90 (solids) | 36.4 | 36.4 | 36.4 | 36.4 | 36.4 | 36.4 |
| Butyl acetate | 3.4 | 1.7 | | 3.4 | 1.7 | |
| Thinner to 28 s DIN 4, 23° | . . . | . . . | . . . | . . . | . . . | . . . |
| C.; Solvesso ® 100/methoxy | | | | | | |
| Propylacetate (1:1) | | | | | | |
| Theoretical SCA content (%) | 0.7 | 0.9 | 1.1 | 0.7 | 0.9 | 1.1 |

Tin plated steel panels of 30×50 cm were coated with a commercial solvent borne primer having an average layer thickness of 40 microns and a water borne pepper red base coat having an average layer thickness of 12-15 microns using a spray robot.

The clear coats were applied with high-speed bell spraying equipment in three different layer thicknesses (20, 30, and 40 microns). During spraying, flash-off, and baking (24 min. at 140° C.) of the clear coats the panels were vertically positioned. The appearance of the clear coats was measured using a commercially available wave scan apparatus and was expressed as GM-tension. The results can be found in the following table.

TABLE IV
GM-Tension

| Formulation | A | B | C | D | E | F |
|---|---|---|---|---|---|---|
| Type of SCA | HDI/BA | HDI/BA | HDI/BA | HY/CHI | HY/CHI | HY/CHI |
| Theoretical % of SCA | 0.7 | 0.9 | 1.1 | 0.7 | 0.9 | 1.1 |
| Layer thickness 20 micron | 7.5 | 8.2 | 7.7 | 7.7 | 8.6 | 8.3 |
| Layer thickness 40 micron | 11.3 | 12.2 | 10.8 | 14.4 | 12.7 | 11.9 |

As can be judged from Table IV, the appearance defined in terms of tension of the clear coats formulated with the HY/CHI SCA has improved in comparison with the HDI/BA SCA at both 20 and 40 microns.

What is claimed is:

1. Essentially acicular crystals of a hydrazine-based diurea derivative having the formula
R′HN—CO—NH—NH—CO—NHR
wherein R and R′ independently are unsubstituted or substituted hydrocarbyl groups and wherein the average length of the crystals is about 0.1 to about 300 micrometers.

2. The hydrazine-based diurea derivative of claim 1 wherein R and R′ are the same.

3. The hydrazine-based diurea derivative of claim 1 wherein the hydrocarbyl group is selected from an alkyl, cycloalkyl, aralkyl, and aryl group.

4. The hydrazine-based diurea derivative of claim 1 wherein the hydrocarbyl group is selected from methyl, butyl, hexyl, octadecyl, benzyl, methoxybenzyl, phenyl, methoxyphenyl, naphthalene, biphenyl, methoxypropyl, ethoxypropyl, methacryloylethyl, and acryloylethyl.

5. An essentially formaldehyde-free coating or adhesive composition comprising the hydrazine-based diurea derivative crystals of claim 1.

6. A rheology modifier for use as a sag control agent or anti-setting agent in coating or adhesive compositions comprising the hydrazine-based diurea derivative crystals of claim 1.

7. An essentially formaldehyde-free thixotropic resin composition comprising the hydrazine-based diurea derivative of claim 1 wherein the hydrazine-based diurea derivative is in needle form.

8. A resin composition according to claim 7 wherein the hydrazine-based diurea derivative is present in a concentrated amount of about 0.1% to about 50% by weight based on the weight of the composition.

9. A process to make coating or adhesive compositions utilizing the resin composition of claim 7 wherein the hydrazine-based diurea derivative is present in a concentrated amount of about 0.1% to about 50% by weight based on the weight of the composition.

10. A process to make an essentially formaldehyde-free thixotropic resin composition comprising a hydrazine-based diurea derivative, comprising the steps of:
i) reacting at least one mono-isocyanate-functional compound of the formula R—NCO, wherein R is an unsubstituted or substituted hydrocarbyl group, with a hydrazine to form a hydrazine-based diurea derivative; and
ii) combining the hydrazine-based diurea derivative with a resin to obtain needle-like crystals of the hydrazine-based diurea derivative embedded in the resin, the needle-like crystals having an average length of from about 0.1 to about 300 micrometers.

11. The process of claim 10 wherein the average length of the needle-like crystals is from about 0.5 to about 100 micrometers.

12. The process of claim 10 wherein the isocyanate and the hydrazine are reacted in the presence of the resin such that the embedded needle-like crystals of the hydrazine-based diurea derivative are obtained in a single step.

13. The process of claim 10 further comprising adding at least one crystal growth modifier to reduce the average length of the crystals to about 0.1 to about 100 micrometers.

14. A process to cure a coating or adhesive composition comprising hydrazine-based diurea derivative crystals according to claim 1 utilizing the application of forced drying and/or baking conditions.
